(19) United States
(12) Patent Application Publication
(10) Pub. No.: US 2024/0349658 A1
(43) Pub. Date: Oct. 24, 2024

(54) EJECTION TRANSPLANTER

(71) Applicant: NUPLANT PTY LTD, Toowong (AU)

(72) Inventors: Robert Teasdale, Toowong (AU); Philip Goodman, Toowong (AU); Ian Davies, Toowong (AU)

(21) Appl. No.: 18/685,560

(22) PCT Filed: Aug. 25, 2022
(86) PCT No.: PCT/AU2022/051030

§ 371 Date: Feb. 22, 2024

(30) Foreign Application Priority Data
Aug. 25, 2021 (AU) 2021221631

(51) Int. Cl. A01G 9/029; A01G 9/08; B25J 9/00; B25J 9/10; B25J 15/00; B25J 15/02

(52) U.S. Cl. CPC A01G 9/0299; A01G 9/088; B25J 9/0084; B25J 9/10; B25J 15/0019; B25J 15/0061; B25J 15/0206

(57) ABSTRACT

A device for robotic transfer of a plantlet from a plantlet holder is provided, the device comprising an opener for opening of the plantlet holder wherein the opener comprises an actuator component for connection to a robotic tool head. Related systems, kits, and methods are further provided.

### FIELD

This invention broadly relates to cultivation of plantlets or plants. More particularly, the invention relates to a device for transfer of tissue cultured plantlets and the like to nursery growth material and the like, although without limitation thereto.

### BACKGROUND

Plant tissue culture typically involves growth of plants under sterile conditions in nutrient medium, usually in glass or plastic containers or similar. Tissue culture is widely used for clonal propagation of plants in both research and commercial contexts. Notably, tissue culture can be used for relatively rapid and large-scale multiplication of commercial plants.

In commercial settings, initial multiplication and growth of plantlets in tissue culture is generally followed by further plant growth in nursery-suitable material. This necessitates transfer of plantlets from tissue culture material, such as agar gel-based or liquid mediums, to material such as potting mix, soil, or other plant growth matrices that may include a range of materials e.g. mineral and polymeric materials such as glass wool, expanded phenolics, or other resins.

Obtaining and handling of individual tissue culture plantlets can present significant challenges. For example, in standard tissue culture arrangements wherein multiple plantlets share a single container, plantlet roots are typically interwoven throughout substantial volumes of culture medium such as agar. Furthermore, conventional arrangements for tissue culture of plants typically do not allow efficient selection of individual plants for transfer based on specific stages of development.

International Patent Publications WO2011/014933 and WO2012/167332, incorporated herein in full by reference, describe plantlet holders and plantlet handling systems that address certain challenges associated with obtaining and handling of tissue culture plantlets. However, there remain significant challenges associated with large-scale, efficient transfer of plantlets propagated in tissue culture medium to other growth mediums, and in particular to nursery-suitable material. New approaches addressing this issue are therefore desirable.

Reference to prior art in the background is not, and should not be taken to be, an acknowledgement that the prior art forms part of the common general knowledge in Australia or in any other country or jurisdiction.

### SUMMARY

A broad form of the present invention relates to a device for releasing a plantlet from a plantlet holder. Suitably, the device is adapted to open a plantlet holder to release a plantlet from the plantlet holder. The device may be further adapted to move a plantlet and/or material in which the plantlet is grown in the plantlet holder to release the plantlet from the plantlet holder.

A first aspect of the invention provides a device for robotic transfer of a plantlet from a plantlet holder, the device comprising an opener for opening of the plantlet holder. The device of the first aspect is connectable to a robot, or a robotic arm thereof. In embodiments, the device is of, connected to, or connectable to a robotic tool head.

Suitably, the opener of the device of the first aspect is adapted to separate at least a portion of the plantlet holder to open the plantlet holder. In embodiments, the opener is adapted to separate a body of the plantlet holder. The opener may be adapted to separate the body of the plantlet holder longitudinally. In embodiments, the opener is adapted to separate the body of the plantlet holder into two substantially even portions along a longitudinal axis thereof.

In embodiments, the opener of the device is adapted to separate the body of the plantlet holder at one or more seams thereof. The one or more seams may be centrally or longitudinally located seams.

In embodiments, the opener of the device is adapted to separate the body of the plantlet holder at one or more seams at or towards a first or front face of the body of the plantlet holder. In embodiments, the opener is adapted to separate the body of the plantlet holder at one or more seams at or towards a second or back face of the body of the plantlet holder. In embodiments, the opener is adapted to separate the body of the plantlet holder at one or more seams at or towards a base or bottom of the plantlet holder.

The opener may be adapted to separate the body of the plantlet holder at one or more openings thereof. The one or more openings may be centrally or longitudinally located openings.

In embodiments, the opener is adapted to separate the body of the plantlet holder at one or more openings at or towards the first or front side of the body. In embodiments, the opener is adapted to separate the body of the plantlet holder at one or more openings at or towards the second or back side of the body. In embodiments, the opener is adapted to separate the body of the plantlet holder at one or more openings at or towards the base or bottom of the plantlet holder.

In embodiments, the opener of the device of the first aspect is adapted to apply a force to the body of the plantlet holder to open the plantlet holder. The force may be a lateral, or substantially lateral, force. In embodiments, the opener is adapted to apply the force, such as the substantially lateral force, to an inner portion of the body of the plantlet holder to separate the body of the plantlet holder. The opener may be adapted to apply the force at or towards the first or front side of the body of the plantlet holder to separate the body of the plantlet holder. The opener may be adapted to apply the force at or towards the second or back side of the body of the plantlet holder to separate the body of the plantlet holder.

In embodiments, the opener of the device is adapted to move a plantlet and/or a growth medium contained in the plantlet holder to release the plantlet from the plantlet holder. Suitably, the opener is adapted to apply a force to the plantlet and/or growth medium to release the plantlet from the plantlet holder. The force may be a pushing force, or the like.

The opener of the device of the first aspect may comprise an actuator component. Suitably, the actuator component of the opener is of, connected to, or connectable to the robotic tool head. Suitably, the actuator component is or comprises an actuator body. The actuator component, or the actuator body thereof, may comprise a frame or bracket, or a portion thereof. The actuator component, or the actuator body thereof, may comprise an actuator member or actuator shaft.

Suitably, the opener of the device of the first aspect comprises a manipulation component for manipulating the plantlet holder. Suitably, the manipulation component is adapted to apply the force, such as the substantially lateral force, to open the plantlet holder, such as to open the plantlet holder by separating the body of the plantlet holder.

In embodiments, the manipulation component of the opener is or comprises an insertion end for insertion into the plantlet holder. The insertion end of the opener may be a thinned and/or tapered insertion end. In embodiments, the insertion end is substantially trapezoidal. In embodiments, the insertion end comprises one or more tapered edges or corners.

Suitably, the manipulation component and/or insertion end of the opener is actuatable via the actuator component of the device. In embodiments, substantially linear actuation of the actuator component can actuate the manipulation component and/or insertion end. In embodiments, substantially linear actuation of the actuator component can apply substantially lateral force to the body of the plantlet holder from the manipulation component and/or insertion end, to thereby open the plantlet holder.

The opener of the device of the first aspect may comprise a blade or tongue component. In embodiments, the blade component is flexible or at least substantially non-rigid. In embodiments, the blade component is substantially rigid.

In embodiments the blade component of the opener is attached or connected to the actuator component of the opener. In embodiments, the blade component is, comprises, or is of, the actuator component of the opener.

In embodiments, the manipulation component and/or insertion end of the opener of the device is of, connected to, or attached to the blade component. In embodiments, the manipulation component and/or insertion end is of an end of the blade component.

In embodiments, the manipulation component and/or insertion end of the opener is of an insertion body. The insertion body may be a substantially elongate insertion body. In embodiments, the insertion body comprises a support portion. The support portion may be opposite the insertion end.

In embodiments, the insertion body comprises a widened central portion adjacent the insertion end.

In embodiments, the insertion body comprises a sloped or slanted insertion face. The insertion face of the insertion body may be sloped or slanted towards the insertion end of the insertion body. The insertion face of the insertion body may be sloped or slanted inwardly, such towards a longitudinal axis of the insertion face.

In embodiments, the insertion body comprises one or more insertion body protrusions. The one or more insertion body protrusions may be insertion body walls. In embodiments, the insertion body comprises at least one side or lateral insertion body wall extending longitudinally at or towards an edge thereof. In embodiments, the insertion body comprise first and second side or lateral insertion body walls extending longitudinally at or towards respective edges, such as left and right edges, thereof.

In embodiments, the one or more walls of the insertion body are ramped or angled walls. In embodiments, the one or more walls ramp down towards the insertion end of the device.

In embodiments of the device comprising an insertion body, suitably, the insertion body is connected or attached to the actuator portion and/or blade portion of the device. In embodiments, the insertion body is in substantially fixed connection with the actuator and/or blade component of the device. In embodiments, the insertion body is in movable connection, such as pivotal or hinged connection, with the actuator and/or blade component of the device.

In embodiments, the insertion portion of the insertion body is substantially aligned with an end of the actuator and/or blade component of the opener of the device. In embodiments, the insertion portion extends beyond the end of the actuator and/or blade component.

The device of the first aspect may comprise a plantlet holder securing component. Suitably, the plantlet holder securing component is adapted to secure the plantlet holder during opening of the plantlet holder with the opener of the device. The securing component may be adapted to secure the plantlet holder during robotic transfer of the plantlet holder.

The securing component may comprise a gripper for gripping the plantlet holder. Suitably, the gripper is actuatable between a gripping configuration and a releasing configuration. Suitably, the gripper is of, connected to, or connectable to the robotic tool head.

Suitably, the gripper of the device of the first aspect is adapted to secure the plantlet holder during opening of the plantlet holder with the opener. The gripper may be adapted to secure the plantlet holder during robotic transfer of the plantlet holder.

Suitably, the gripper of the device comprises one or more gripping portions for gripping the plantlet holder. In embodiments, the gripper comprises first and second gripping portions. The gripping portions of the gripper may be adapted to secure a handling portion of the plantlet holder. In embodiments, the first and second gripping portions are shaped to fit the handling portion of the plantlet holder therebetween.

In embodiments, the gripper comprises first and second fingers. Suitably, the first and second fingers are connected or connectable to the robotic tool head. The first and second gripping portions of the gripper may be of or connected to the first and second fingers. In embodiments, the first and second gripping portions are located at or towards respective ends of the first and second fingers.

Suitably, the first and second gripping portions and/or fingers of the gripper are positioned together, or relatively together, in the gripping configuration, and positioned apart, or relatively apart, in the releasing configuration.

Suitably, the gripper of the device is adapted to position the plantlet holder relative to the manipulation or insertion portion of the opener of the device, wherein actuation of the opener can open the plantlet holder gripped by the gripper.

In embodiments, a central or longitudinal axis of the gripper is substantially alignable to a central or longitudinal axis of the opener. In embodiments, the fingers of the gripper can grip the plantlet holder wherein the plantlet holder is substantially aligned with the longitudinal axis of the gripper and the longitudinal axis of the actuator and/or blade component of the opener.

In embodiments, linear actuation of the actuator component of the opener of the device is adapted to apply force from the manipulation or insertion portion of the opener to the plantlet holder gripped by the gripper of the device, to open the plantlet holder.

The device of the first aspect may comprise one or more guides, for guiding the opener, or a portion thereof, relative to the plantlet holder. Suitably, the one or more guides are for guiding the opener, or the portion thereof, relative to the plantlet holder when held by the gripper. The one or more guides may be adapted to guide the insertion portion of the device against the gripper.

In embodiments, the device comprises a guide portion comprising one or more protrusions for contacting the gripper to guide the insertion portion relative to the plantlet holder. Suitably, actuation, such as linear actuation, of the actuator component of the opener forces the one or more protrusions of the guide portion against the gripper to guide the insertion portion relative to the plantlet holder.

The one or more protrusions of the guide portion may be ramped or angled protrusions. In embodiments, actuation, such as linear actuation, of the actuator portion of the opener forces the one or more ramped protrusions against the gripper wherein the insertion portion of the opener is forced away from the gripper and guided relative to the plantlet holder held by the gripper.

In embodiments, the guide portion comprises a central protrusion. The central protrusion may be for contacting a central part of the first and/or second gripping portions.

In embodiments, the guide portion of the device comprises first and second spaced protrusions. The first and second spaced protrusions may be for contacting lateral parts of the first and second gripping portions of the gripper, respectively.

In embodiments, one or more of the guides of the device, or guide portions thereof, are of or attached to the insertion body of the device. In embodiments, the central protrusion of the guide portion is of the insertion body of the device.

In embodiments, one or more of the guides of the device, or guide portions thereof, are of or attached to the blade portion of the device. In embodiments, the first and second spaced protrusions of the guide portion are of the blade portion of the device.

In embodiments wherein the device comprises a guide portion comprising a protrusion of the insertion body, the device may comprise a blade portion comprising one or more slots or channels through which the protrusion extends or can extend.

In an embodiment, the device of the first aspect is a device for connection to a robot tool head of a plantlet handling system, the device comprising:
an opener for opening a plantlet holder, the plantlet holder comprising a body comprising one or more central seams and/or central openings and a handling portion; and
a gripper for securing the plantlet holder during opening of the plantlet holder with the opener, wherein
the opener comprises an actuator component for connection to the robot tool head and a tapered insertion portion connected to the actuator, wherein the tapered insertion portion is for insertion into the body of the plantlet holder by actuation of the actuator portion, to separate the plantlet holder body along the one or more central seams and/or central openings thereof, and
the gripper comprises first and second fingers for connection to the robot tool head, wherein the gripper is actuatable between a gripping configuration wherein gripping portions of the first and second fingers are together for securing the handling portion of the plantlet holder, and a releasing configuration wherein gripping portions of the first and second fingers are apart for releasing the handling portion of the plantlet holder.

The device may further comprise a guide for guiding the opener against the gripper for opening of the plantlet holder, wherein the guide comprises at least one protrusion for contacting the gripping portion of the first and/or second fingers of the gripper, wherein the actuation of the actuator portion of the opener forces the protrusion against the gripping portion to guide the insertion portion of the opener away from the gripper for insertion into the body of the plantlet holder.

A second aspect of the invention provides a robot tool head comprising one or more devices of the first aspect. In embodiments of the second aspect, the actuator component of each of the one or more devices of the first aspect is connected to a respective actuator of the robot tool head. The actuator, or each of the respective actuators, of the robot tool head may be a pneumatic actuator.

A third aspect of the invention provides a robotic system comprising one or more devices of the first aspect. Suitably, the device of the first aspect is of or connected to a robot tool head of the robotic system. In embodiments, the robotic tool head is connected to a robotic arm of the robotic system.

In embodiments, the robotic system of the third aspect comprises, or is connected, connectable, or otherwise associated with a source of plantlets contained in plantlet holders. In embodiments, the plantlets are tissue cultured plantlets, or the like. Suitably, the tissue cultured plantlets are in tissue culture media, such as gel or liquid media. The source of plantlets contained in plantlet holders may comprise a tray or housing for supporting a plurality of the plantlet holders.

In embodiments, the robotic system comprises, or is connected, connectable or otherwise associated with a source of transplant material. The transplant material may be nursery suitable material or the like, such as potting mix, soil, or an artificial or semi-artificial growth matrix. The source of transplant material may comprise a tray or housing for receiving a plurality of plantlets from the plantlet holders.

A fifth aspect of the invention provides a system comprising one or more devices of the first aspect; and one or more plantlet holders connected with the one or more devices of the first aspect.

A sixth aspect of the invention provides a kit comprising one or more devices of the first aspect; and one or more plantlet holders.

Suitably, the plantlet holder according to the fifth and sixth aspects comprises an opening, such as an upper or top opening, into which the opener of the device of the first aspect can be inserted. Suitably, the plantlet holder according to the fifth and sixth aspects comprises a body comprising one or more central seams and/or central openings for separation by the opener of the device of the first aspect.

Suitably, the plantlet holder according to the fifth and sixth aspects comprises one or more portions adapted for engagement with the opener of the device of the first aspect. In embodiments, the plantlet holder comprises one or more portions adapted for engagement with the insertion end and/or insertion body of the opener of the device of the first aspect.

In embodiments, the opening of the plantlet holder, such as the upper or top opening, comprises one or more surfaces adapted for engagement with the opener of the first aspect. The one or more surfaces may be bevelled surfaces and/or corner surfaces. In embodiments, the surfaces, such as the bevelled surfaces and/or corner surfaces, are at or towards a first or front side of the body of the plantlet holder. In embodiment, the surfaces, such as the bevelled surfaces and/or corner surfaces, are at or towards a second or rear side of the body of the plantlet holder.

A seventh aspect of the invention provides a plantlet holder for, or suitable for, the system of the fifth aspect or the kit of the sixth aspect.

Suitably, the plantlet holder according to the seventh aspect comprises an opening, such as an upper or top opening, into which the opener of the device of the first aspect can be inserted. Suitably, the plantlet holder according to the seventh aspect comprises a body comprising one or more central seams and/or central openings for separation by the opener of the device of the first aspect.

Suitably, the plantlet holder according to the seventh aspect comprises one or more portions adapted for engagement with the opener of the device of the first aspect. In embodiments, the plantlet holder comprises one or more portions adapted for engagement with the insertion end and/or insertion body of the opener of the device of the first aspect.

In embodiments, the opening of the plantlet holder, such as the upper or top opening, comprises one or more surfaces adapted for engagement with the opener of the first aspect. The surfaces may be bevelled surfaces and/or corner surfaces. In embodiments, the surfaces, such as the bevelled surfaces and/or corner surfaces, are at or towards a first or front side of the body of the plantlet holder. In embodiment, the surfaces, such as the bevelled surfaces and/or corner surfaces, are at or towards a second or rear side of the body of the plantlet holder.

An eighth aspect of the invention provides a method of robotically transferring a plantlet from a plantlet holder, including a step of opening the plantlet holder with an opener of a robotic device. Suitably the robotic device is of or connected to a robotic tool head or the like.

The method of the eighth aspect may include a step of moving the plantlet and/or material in which the plantlet is grown with the opener to release the plantlet from the plantlet holder.

In embodiments, the plantlet held by the plantlet holder according to the method of the eighth aspect is a tissue cultured plantlet, or the like. Suitably, the plantlet is grown in tissue culture media, or the like. In embodiments, the tissue culture media is a gel-based media. In embodiments, the tissue culture media is a liquid media.

In embodiments, the plantlet holder according to the method of the eighth aspect comprises a body including one or more seams and/or openings. The seams and/or openings may be longitudinal or central seams or openings.

In embodiments, the one or more seams or openings of the plantlet holder include seams or openings at or towards a first or front side of the body of the plantlet holder. In embodiments, the one or more seams or openings of the plantlet holder include seams or openings at or towards a second or back side of the body of the plantlet holder. In embodiments, the one or more seams or openings include seams or openings at or towards a bottom or base of the plantlet holder.

In embodiments, the step of opening the plantlet holder according to the method of the eighth aspect includes applying a force to the plantlet holder with the opener to separate the plantlet holder. The force may be a lateral, or substantially lateral, force. The force may be applied to an inner portion of the body of the plantlet holder. In embodiments, the force is applied at or towards the second or back side of the plantlet holder. In embodiments, the force is applied at or towards the first or back side of the plantlet holder.

In embodiments, the step of moving the plantlet and/or material in which the plantlet is grown with the opener includes applying a pushing force, or the like, to the plantlet and/or material.

In embodiments, the plantlet holder according to the method of the eighth aspect comprises a handling portion. The handling portion may be located at or towards a second or back side of the body of the plantlet holder. In embodiments, the handling portion comprises an extension or protrusion at or toward an upper part of the back side of the body of the plantlet holder.

The method of the eighth aspect may include a step of gripping the plantlet holder with a gripper of the device. In embodiments, the step of gripping the plantlet holder with the gripper of the device includes gripping the handling portion of the plantlet holder with the gripper.

Suitably, the step of gripping the plantlet holder with the gripper includes gripping the plantlet holder while opening the plantlet holder with the opener. In embodiments, the step of gripping the plantlet holder with the gripper includes gripping the plantlet holder while moving the plantlet and/or material in which the plantlet is grown with the opener.

The method of the eighth aspect may include a step of collecting the plantlet from a source of plantlets contained in plantlet holders. The source of plantlets containing the plantlet holders may comprise a tray or housing for supporting the plurality of plantlet holders.

Suitably, the step of collecting the plantlet includes positioning the device near to the plantlet holder containing the plantlet. The step of collecting the plantlet may include positioning the device relative to a specific cell of the tray or housing for supporting the plantlet holders. In embodiments, the step of positioning the device includes positioning the device with a robotic arm of a robotic system connected to the device. The step of collecting the plantlet may include gripping the plantlet holder containing the plantlet with the gripper of the device.

The method of the eighth aspect may include a step of placing the plantlet into transplant material. The transplant material may be nursery-suitable material, such as potting mix, soil, or an artificial or semi-artificial growth matrix. The source of transplant material may comprise a tray or housing for receiving a plurality of plantlets from the plantlet holders.

Suitably, the step of placing the plantlet into transplant material includes positioning the device near to the transplant material. The step of placing the plantlet into the transplant material may include positioning the device relative to a specific cell of the tray or housing for receiving the plurality of plantlets from the plantlet holders. In embodiments, the step of positioning the device includes positioning the device with the robotic arm of the robotic system connected to the device.

In embodiments, the step of placing the plantlet into the transplant material includes preparing an indentation or groove, or the like, in the transplant material into which the plantlet is placed. The indentation or groove may be prepared by forcing the body of the plantlet holder against the transplant material under control of the robotic arm and/or robot tool head of the robotic system. Suitably, the plantlet holder is gripped by the gripper of the device when forced against the transplant material.

The method of transferring the plantlet from the plantlet holder according to the eighth aspect may be a method of concurrently transferring a plurality of plantlets from a plurality of plantlet holders, including a step of concurrently opening each of the plurality of plantlet holders with a respective plurality of openers.

In embodiments, the method of the eighth aspect may include a step of adjusting spacing of the plurality of openers to align with a plurality of cells of the tray or housing comprising the plurality of plantlet holders. In embodiments, the method of the eighth aspect may include a step of adjusting spacing of the plurality of openers to align with a plurality of cells of the tray or housing for receiving the plurality of plantlets.

In embodiments, the device according to the method of the eighth aspect is the device of the first aspect.

In embodiments, the system according to the method of the eighth aspect is the system of the third aspect.

In embodiments, the plantlet holder according to the method of the eighth aspect is the plantlet holder of the seventh aspect.

### BRIEF DESCRIPTION OF THE DRAWINGS

The invention will be described hereinafter with reference to typical embodiments illustrated in the drawings, wherein:

FIG. 1 sets forth an embodiment of plantlet holder 1. FIG. 1A is a front perspective view of the plantlet holder in a closed configuration. FIG. 1B is an upper side perspective view of the plantlet holder in an open configuration.

FIG. 2 sets forth another embodiment of plantlet holder 1. FIG. 2A is a front perspective view of the plantlet holder in a closed configuration. FIG. 2B is an upper side perspective view of the plantlet holder in an open configuration.

FIG. 3 sets forth a front perspective view of another embodiment of plantlet holder 1.

FIG. 4 sets forth a front perspective view of another embodiment of plantlet holder 1.

FIG. 5 sets forth a side view and a front perspective view of device 100. Device 100 is an embodiment of a device for robotic transfer of a plantlet from a plantlet holder. In FIG. 5, device 100 is engaged with the plantlet holder of FIG. 1, wherein the plantlet holder is holding tissue cultured plantlet 2 grown in plug of agar medium 3, and opener 110 of device 100 is positioned away from the plantlet holder.

FIG. 6 sets forth a side view and a front perspective view of the device of FIG. 5, wherein opener 110 is positioned towards the plantlet holder.

FIG. 7 sets forth a side view and a front perspective view of the device of FIG. 5, wherein opener 110 has separated body 10 of the plantlet holder and displaced the tissue cultured plantlet and agar plug from the plantlet holder.

FIG. 8 sets forth a rear perspective view of the device of FIG. 5. In FIG. 5, gripper 150 is in a closed configuration.

FIG. 9 sets forth (A) a cross-sectional view of the device of FIG. 5 showing fin 1405 of insertion body 140 of the opener of the device; and (B) a perspective view of a bracket comprising actuator component 120 and blade component 130. The bracket comprising actuator component 120 and blade component 130 may be considered separate components or combined actuator and blade component 125.

FIG. 10 sets forth a front perspective view of a plurality of devices of FIG. 5 when connected to a robotic tool head (not shown). In FIG. 10 the respective plantlet holders have been obtained from tissue culture tray 4. The plurality of plantlet holders are in a narrowed configuration corresponding to cell spacing of tissue culture tray 4.

FIG. 11 sets forth a rear perspective view of a plurality of devices of FIG. 5 when connected to a robotic tool head (not shown) and in the narrowed configuration as for FIG. 10.

FIG. 12 sets forth a front perspective view of a plurality of devices of FIG. 5 when connected to a robotic tool head (not shown). In FIG. 12 the respective plantlet holders are arranged for transfer of the respective plantlets to nursery tray 5. The plurality of plantlet holders are in a widened configuration corresponding to cell spacing of nursery tray 5.

FIG. 13 sets forth a front perspective view of the device of FIG. 5, wherein the device has displaced the tissue culture plantlet and agar plug into a cell of a nursery tray. In FIG. 13, the cell of the nursery tray is shown in cross-section, wherein indentation 6 (formed by the plantlet holder under robotic control using the device) is visible.

FIG. 14 sets forth a lower front perspective view of robot tool head 50, with cover removed showing actuating mechanisms, comprising a plurality of the devices of FIG. 5.

FIG. 15 sets forth an upper perspective view of robotic system 1000 comprising the robotic tool head of FIG. 14 connected to robotic arm 500, the system operably associated with a plurality of tissue culture trays 4 and a plurality of nursery trays 5. In FIG. 15, the robotic tool head is positioned over a tissue culture tray after removal of a plurality of plantlet holders from the tissue culture tray.

FIG. 16 sets forth an upper perspective view of the system of FIG. 15, wherein the robotic tool head is positioned in transition between tissue culture trays 4 and nursery trays 5.

FIG. 17 sets forth an upper perspective view of the system of FIG. 15, wherein the robotic tool head is positioned over nursery tray 5 prior to delivery of plantlets and into the nursery tray.

FIG. 18 sets forth a front view of device 200. Device 200 is another embodiment of a device for robotic transfer of a plantlet from a plantlet holder. In FIG. 18, a plurality of devices 200 are connected to a robotic tool head (not shown) and engaged with a respective plurality of plantlet holders of FIG. 1. The plantlet holders are holding tissue cultured plantlets grown in plugs of agar medium, and respective openers 210 of device 200 are positioned away from the plantlet holders.

FIG. 19 sets forth a front perspective view of the devices of FIG. 18, wherein the respective plantlet holders have been obtained from tissue culture tray 4. The plurality of plantlet holders are in a narrowed configuration corresponding to cell spacing of tissue culture tray 4.

FIG. 20 sets forth a rear perspective view of the devices of FIG. 18, wherein opener 210 has separated body 10 of the plantlet holder and displaced the tissue cultured plantlet and agar plug from the plantlet holder for transfer of the respective plantlets into nursery tray 5. The plurality of plantlet holders are in a widened configuration corresponding to cell spacing of nursery tray 5.

FIG. 21 sets forth a side view of the arrangement of FIG. 20.

FIG. 22 sets forth a front perspective view of device 300. Device 300 is another embodiment of a device for robotic transfer of a plantlet from a plantlet holder. In FIG. 22, a plurality of devices 300 are connected to a robotic tool head and engaged with a respective plurality of plantlet holders of FIG. 1. The plantlet holders are holding tissue cultured plantlets grown in plugs of agar medium, and respective openers 310 of device 300 are positioned away from the plantlet holders.

FIG. 23 sets forth a side view and a perspective view of the device of FIG. 5 when ejecting a plantlet and agar plug from the plantlet holder of FIG. 4.

FIG. 24 sets forth a front perspective view of device 400. Device 400 is another embodiment of a device for robotic transfer of a plantlet from a plantlet holder. In FIG. 24, device 400 is engaged with the plantlet holder of FIG. 1, wherein opener 410 is positioned away from the plantlet holder.

FIG. 25 sets forth (A) a side perspective view; (B) a front perspective view; and (C) a transverse cross-sectional view of the device of FIG. 24.

FIG. 26 sets forth (A) a front view; and (B) a rear view of the device of FIG. 24. In FIG. 26, the device is engaged with the plantlet holder of FIG. 1, wherein the plantlet holder is holding tissue cultured plantlet 2 grown in plug of agar medium 3, and opener 410 is positioned away from the plantlet holder.

FIG. 27 sets forth a cross-sectional view of the device of FIG. 24. In FIG. 27, opener 410 is positioned partly inserted into the plantlet holder.

FIG. 28 sets forth a front perspective view of a plurality of devices of FIG. 24 when connected to a robotic tool head (not shown) and in a narrowed configuration. In FIG. 28, openers 410 of the respective devices are positioned away from respective plantlet holders of FIG. 1.

FIG. 29 sets forth a front perspective view of a plurality of devices of FIG. 24 when connected to a robotic tool head (not shown) and in a widened configuration. In FIG. 29, openers 410 have separated body 10 of the plantlet holders and displaced the tissue cultured plantlets and agar plugs from the plantlet holders.

FIG. 30 sets forth a front view of a plurality of devices of FIG. 24 when connected to a robotic tool head (not shown) and in a widened configuration. In FIG. 30, openers 410 of the respective devices are positioned away from respective plantlet holders of FIG. 1.

FIG. 31 sets forth a front view of a plurality of devices of FIG. 24 when connected to a robotic tool head (not shown) and in a widened configuration. In FIG. 31, openers 410 have separated body 10 of the plantlet holders and displaced the tissue cultured plantlets and agar plugs from the plantlet holders.

FIG. 32 sets forth an upper perspective view of robot tool head 50 comprising a plurality of devices of FIG. 24 and associated with tissue culture tray 4. In FIG. 32 the respective plantlet holders have been obtained from tissue culture tray 4. The plurality of plantlet holders are in a narrowed configuration corresponding to cell spacing of tissue culture tray 4.

FIG. 33 sets forth a close-up perspective view of robot tool head 50 comprising a plurality of devices of FIG. 24 and associated with nursery tray 5. In FIG. 33, openers 410 have separated body 10 of plantlet holders 1 and are displacing the tissue cultured plantlets 2 and agar plug 3 from the plantlet holders 1 for transfer of the respective plantlets into nursery tray 5. The plurality of plantlet holders are in a widened configuration corresponding to cell spacing of nursery tray 5.

### DESCRIPTION OF EMBODIMENTS

Described herein are embodiments of a device for robotic transfer of plantlets from plantlet holders. More particularly, the device is adapted for transfer of tissue culture plantlets from holders adapted for individual plantlet handling. Reference will primarily be made to one typical embodiment of the device, device 100. Alternative embodiments of the device, device 200, device 300, and device 400, will also be described.

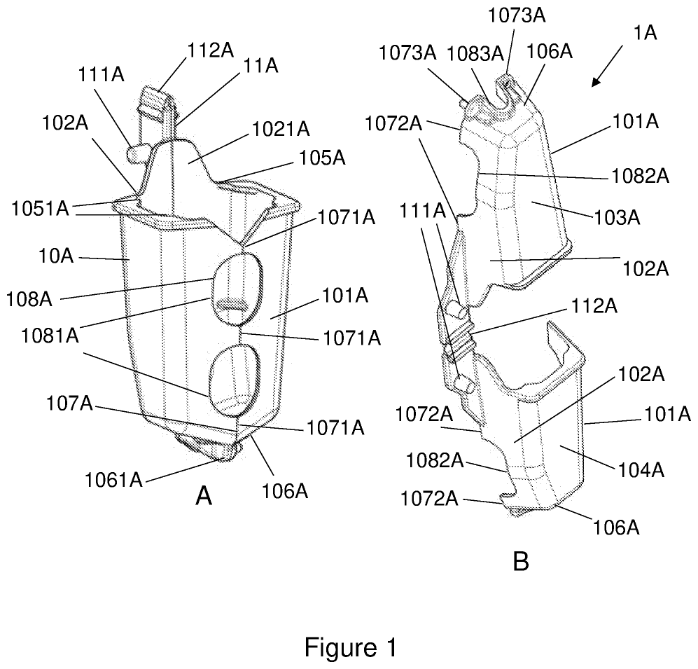

FIGS. 1 to 4 show certain typical embodiments of plantlet holders that can be opened using devices 100-400. Plantlet holders as illustrated in FIGS. 1 to 4 are referred to generally herein as plantlet holder 1. As further described below, typical embodiments of plantlet holder 1 may be the same or similar as disclosed in International Patent Publication WO2011/014933, incorporated in full by refence. The specific embodiments of plantlet holder 1 shown in FIGS. 1, 2, 3, and 4 will at times respectively be referred to as plantlet holder 1A, plantlet holder 1B, plantlet holder 1C, and plantlet holder 1D.

Generally, plantlet holder 1 comprises body 10 (10A, 10B, 10C, 10D); and handling portion 11 (11A, 11B, 11C, 11D). Plantlet holder body 10 is a separable body comprising front face 101 (101A, 101B, 101C, 101D); rear face 102 (102A, 102B, 102C, 102D); left side 103 (103A, 103B, 103C, 103D); right side 104 (104A, 104B, 104C, 104D); top 105 (105A, 105B, 105C, 105D); and base 106 (106A, 106B, 106C, 106D). Body 10 of plantlet holder 1 is adapted for longitudinal separation into halves comprising left side 103 and right side 104, respectively. Longitudinal separation of body 10 is facilitated by one or more central seams 107 (107A, 107B, 107C, 107D) and/or openings 108 (108A, 108B, 108C, 108D).

Handling portion 11 of plantlet holder 1 extends from shoulder portion 1021 (1021A, 1021, B, 1021C, 1021D) of rear face 102. Handling portion 11 may comprise lateral projections 111 (111A, 111B, 111C). Handling portion 11 may further comprise hinge arrangement 112 (112A, 112B, 112C).

As shown in FIG. 1, plantlet holder 1A comprises plantlet holder body 10A. Front face 101A of body 10A comprises front face seam portions 1071A and upper and lower front face openings 1081A. Rear face 102A comprises rear face seam portions 1072A and rear face opening 1082A. Base 106A comprises base seam portions 1073A and base opening 1083A. Connection between halves of body 10A is facilitated by pin and hole arrangement 1061A of base 106A.

Plantlet holder 1A further comprises handling portion 11A, extending from shoulder portion 1021A of rear face 102A. Handling portion 11A comprise lateral projections 111A; and upper hinge 112A.

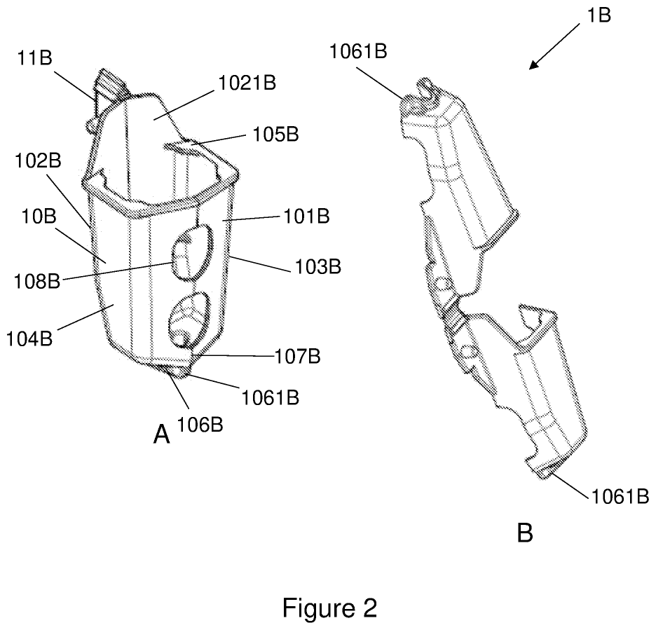

As shown in FIG. 2, plantlet holder 1B has a similar structure to plantlet holder 1A. Notably, shoulder portion 1021B is increased in size relative to shoulder portion 1021A. Notably, base 106B comprises notch and tongue arrangement 1061B in place of pin and hole arrangement 1061A.

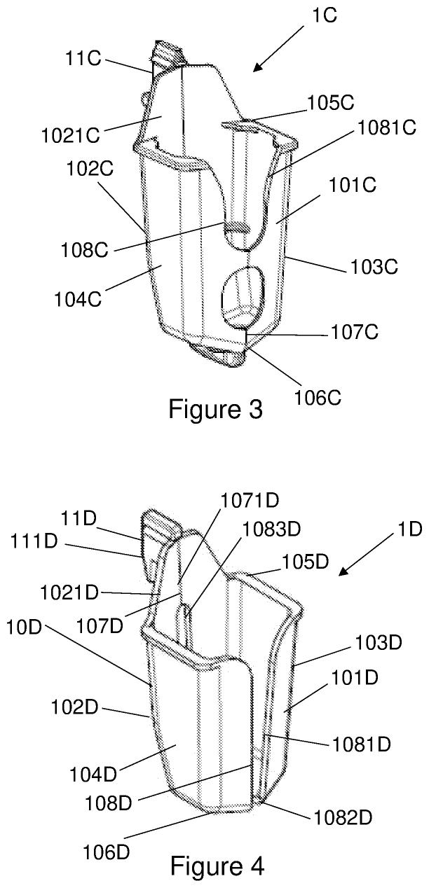

As shown in FIG. 3, plantlet holder 1C has a similar structure to plantlet holder 1A and plantlet holder 1B. Notably, upper front face opening 1081C widens to a top edge of front face 101, to facilitate basal tissue culture dissections in plantlet holder 1C.

As shown in FIG. 4, plantlet holder 1D comprises plantlet holder body 10D. Front face 101D of body 10D comprises longitudinal opening 1081D extending along the full length thereof and transitioning into base opening 1082D. Rear face 102D comprises upper and lower seam portions 1071D and central elongated opening 1083D. Handling portion 11D comprises hook 111D in place of lateral projections as described for plantlet holders 1A-1C.

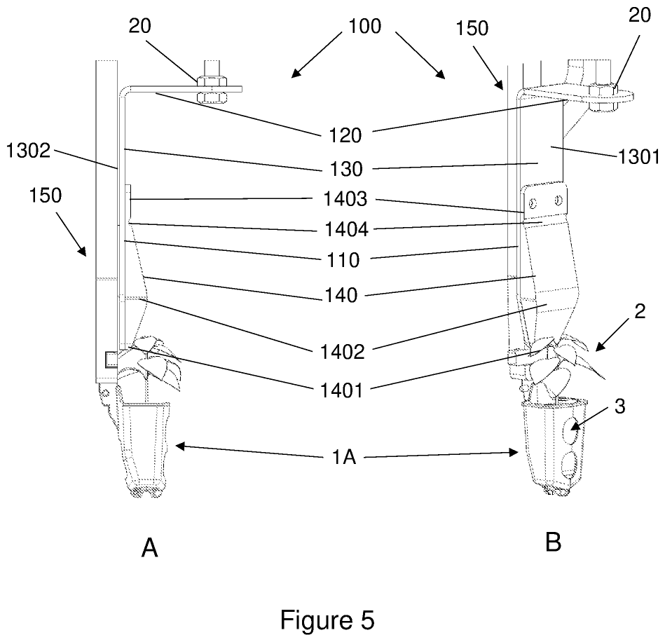

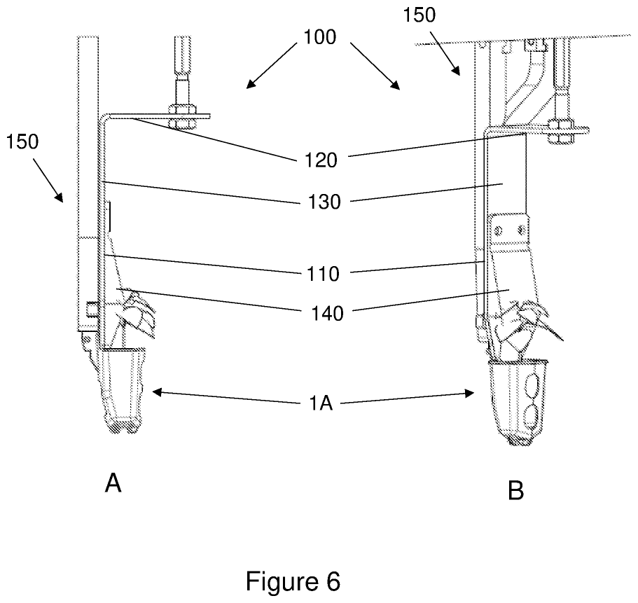

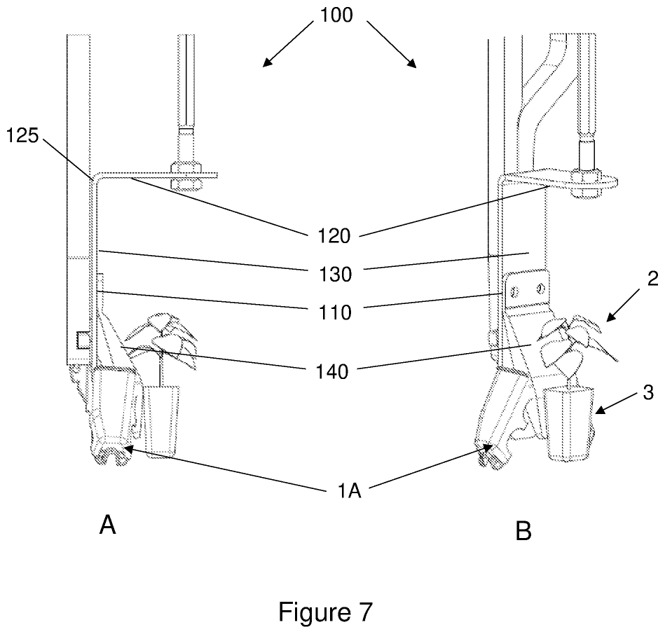

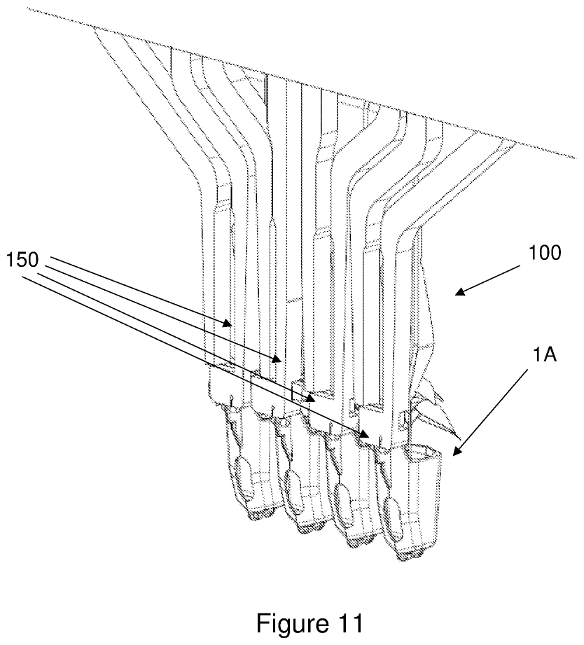

FIGS. 5 to 13 show device 100. As explained in further detail below, device 100 is shown holding plantlet holder 1A in grippers thereof. It will be appreciated, however, that plantlet holder 1A is not part of device 100 itself. It will be further appreciated that, while device 100 typically interacts with a securing component of some form for securing a plantlet holder during opening of the plantlet holder, the securing component generally, and the grippers specifically, are optional components of device 100. Similar considerations apply in relation to devices 200,300, and 400 described hereinbelow.

Device 100 comprises opener 110 connectable to a robot tool head or the like, for opening a plantlet holder such as plantlet holder 1A, 1B, 1C, or 1D hereinabove described.

Opener 110 of device 100 comprises actuator component 120; blade component 130; and insertion body 140.

Actuator component 120 and blade component 130 of opener 110 are formed as an L-shaped bracket, wherein actuator component 120 comprises a horizontal portion of the bracket and blade component 130 comprises a vertical portion of the bracket. To avoid doubt, it will be understood that components 120/130 of device 100 may be considered separate components or combined actuator and blade component 125.

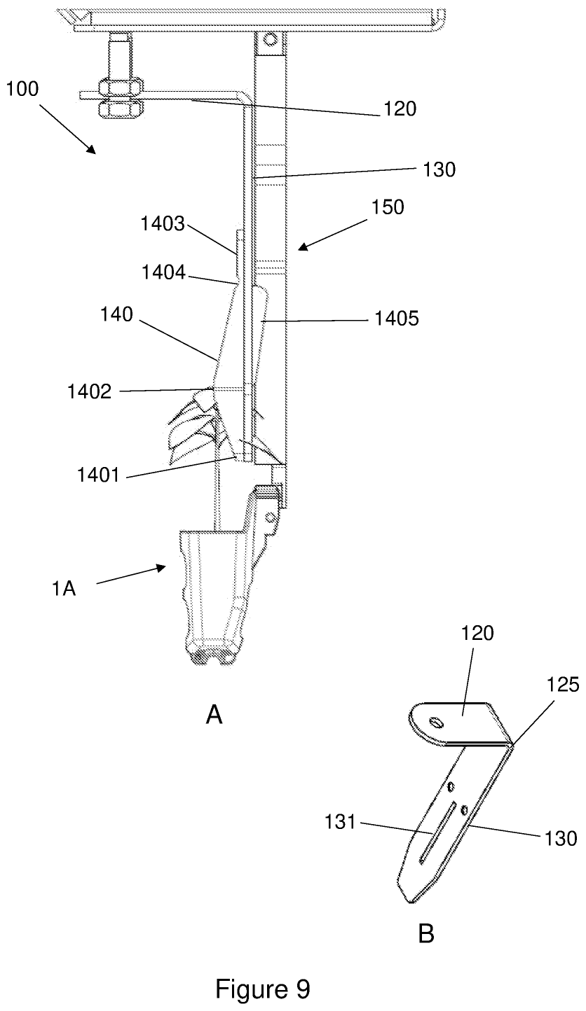

Actuator component 120 is attachable to bolt arrangement 20 for connection of device 100 to the robot tool head. Blade component 130 comprises first or front face 1301; and second or back face 1302. Blade component 130 extends away from the robot tool head towards a position of plantlet holder 1A. Typically, blade component 130 comprises slit 131 as best seen in FIG. 9, or alternatively a forked end, to accommodate fin of insertion body 140 as further described below.

As best seen in FIG. 9A, insertion body 140 of opener 110 comprises insertion end 1401; central portion 1402; and support portion 1403. Insertion body 140 has a arcuate longitudinal profile. Central portion 1402 is substantially thickened relative to insertion end 1401 and support portion 1403. Insertion end 1401 is also somewhat inwardly tapered relative to central portion 1402.

Support portion 1403 of insertion body 140 is fixed to front face 1301 of blade component 130, such as by mechanical fasteners and/or chemical adhesive. Hinge portion 1404 joins support portion 1403 to the remainder of insertion body 140. Insertion body 140 is pivotable away from front face 1301 of blade component 130 via hinge portion 1404.

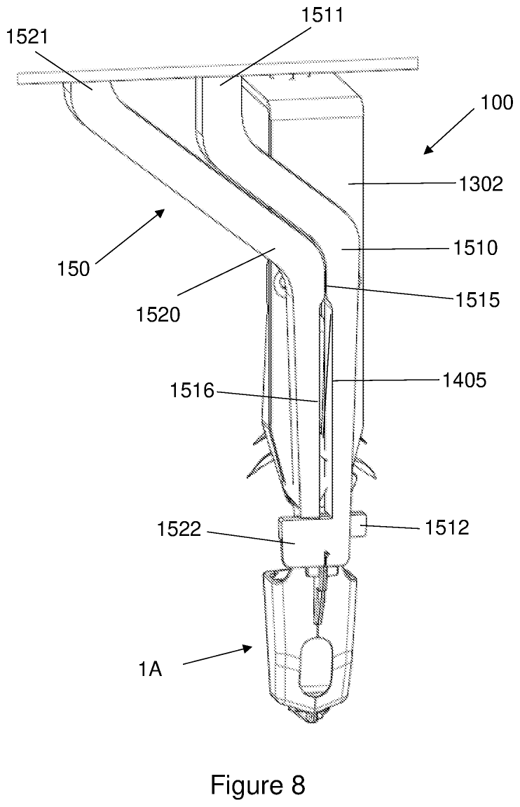

As best seen in FIGS. 8 and 9, insertion body 140 of opener 110 further comprises fin 1405 extending from insertion body 140 in the direction of blade component 130. Fin 1405 is a triangular fin, a front tip thereof joining insertion body 140 towards insertion end 1401. It will be appreciated that fin 1405 can pass through slit 131 of blade component 130 such that insertion body 140 can rest against front face 1301 of blade component 130, as clearly shown in FIG. 5. As hereinbelow described, fin 1405 functions as a guide for guiding insertion body 140 and insertion end 1401 thereof into plantlet holder 1.

Opener 110 of device 100 is associated with gripper 150. To avoid doubt, it will be appreciated that gripper 150 may, but need not, be considered part of device 100.

As best seen in FIG. 8, gripper 150 comprises first finger 1510; and second finger 1520. Respective connection ends of 1511 and 1521 of first finger 1510 and second finger 1520 are connectable to the robot tool head (not shown in FIG. 8). First finger 1510 and second finger 1520 further comprise respective gripping ends 1512 and 1522, opposite connection ends 1511 and 1521.

Gripping ends 1512 and 1522 of first finger 1510 and 1520, respectively, are slidably engaged or slidably engageable. Gripper 150 may comprise central abutment arrangement 1515 of first finger 1510 and second finger 1520, wherein slit 1516 between first finger 1510 and second finger 1520 is maintained when first finger 1510 and second finger 1520 are fully engaged.

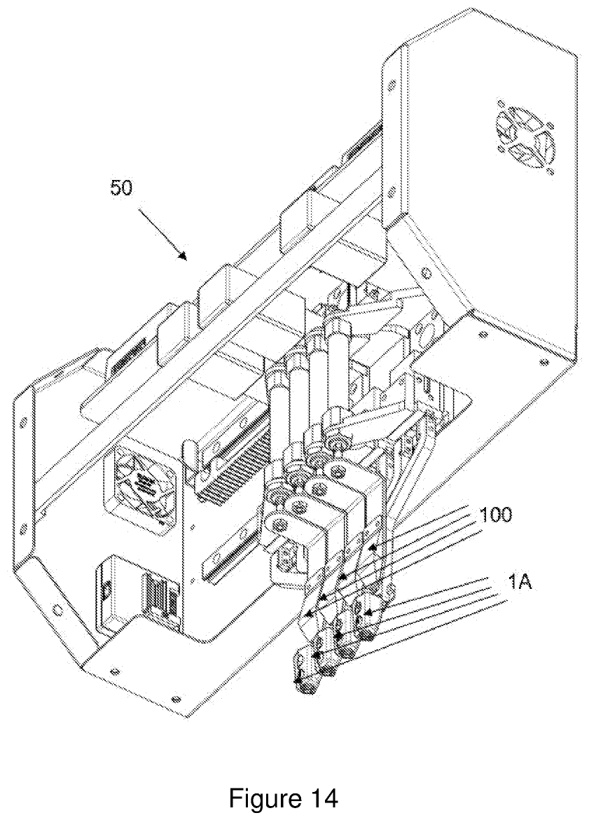

FIG. 14 shows robot tool head 50 comprising a plurality of devices 100 attached thereto. In FIG. 14, a cover of robot tool head 50 is removed, showing internal components and connections of the robot head. As shown in FIG. 14, actuator component 120 of each respective device 100 is connected a respective pneumatic actuator of robot tool head 50 via a respective bolt arrangement 20. Additionally, connection ends 1511 and 1521 of respective grippers 150 are attached to actuation machinery of robot tool head 50 for actuating respective grippers 150. Pneumatic actuator and gripper actuator machinery is well-known in the art of robotics, and the specific structure of these components of robot tool head is not dealt with in detail here.

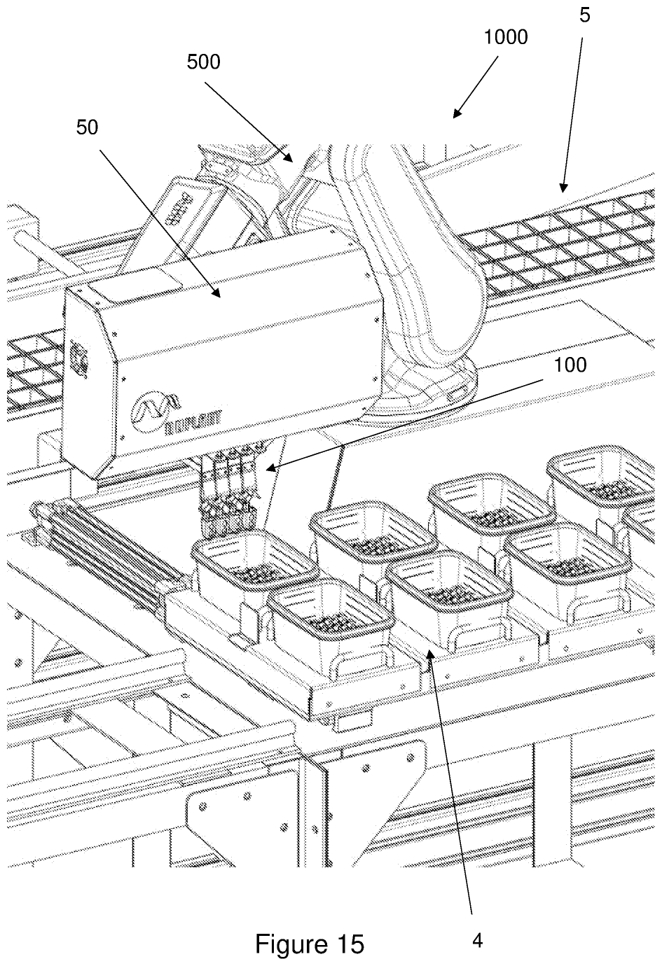

FIGS. 15 to 17 show robotic system 1000 comprising robot arm 500 attached to robot tool head 50 comprising four devices 100. Robotic system 1000 is located at a workstation comprising a plurality of tissue culture trays 4 and a plurality of nursery trays 5. Robotic arm machinery and the use of robotic arms in the context of workstations is well-known in the art of robotics, and the specific structure of these components is not dealt with in detail here.

Typical use of device 100 will now be described in the context of robotic systems such as system 1000. It will be understood that operation of robotic systems, such as system 1000, is suitably controlled by robot computing operating systems as are well-known in the art. Robotic operation is typically also guided by spatial sensors and the like, as are well-known in the art of robotics.

Typically, although not exclusively, a plurality of devices 100 are used in coordinated fashion with a robotic system such as system 1000. It will be appreciated that the particular number of devices 100 used in coordination will relate to the capacity of the robotic system (or robot tool head thereof) and the structure (such as cell arrangement) of trays (typically tissue culture and nursery material trays, or the like) with which the robotic system is used. As depicted in FIGS. 15-17, four devices 100 are used in coordination in system 1000, although it will be fully understood that this is exemplary only.

To prepare system 1000 for use, each device 100 is connected to robot head 50. More particularly, actuator component 120 of each respective device 100 is connected to a respective pneumatic actuator of robot head 50 via a respective bolt arrangement 20. Additionally, a respective gripper 150 is connected to actuation machinery of robot head 50 for actuating respective grippers 150.

In use, referring to FIG. 15, robotic arm 500 positions robot head 50 comprising respective devices 100 over specific cells of specific tissue culture tray 4, tissue culture tray 4 comprising individual plantlet holders 1, and individual plantlet holders 1 comprising individual plantlets 2 in growth medium such as agar plugs 3.

In use, movement of robotic arm 500 in combination with actuation of grippers 150 facilitates secure gripping of handling portion 11 of respective plantlet holders 1 in respective grippers 150.

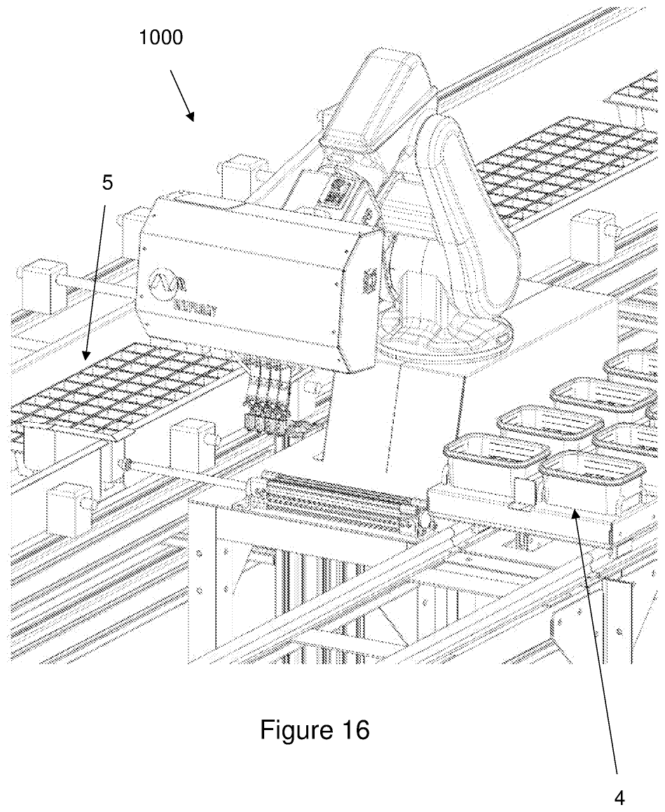

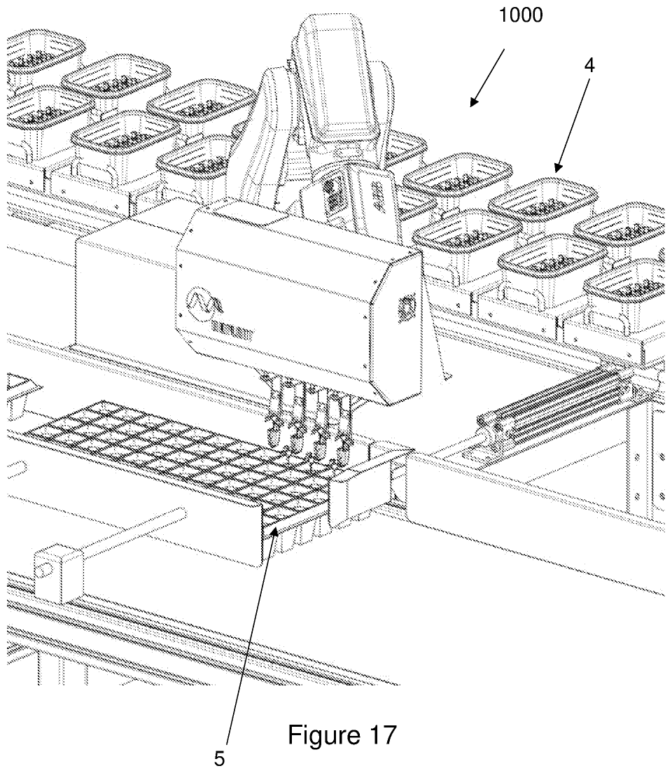

In use, referring to FIG. 16 and FIG. 17, with respective plantlet holders 1 securely gripped in respective gripper 150, robotic arm 500 moves respective devices 100 and associated respective plantlet holders 1 over specific cells of specific nursery tray 5. Cells of nursery tray 5 comprise a nursery growth medium such as potting mix, soil, or another suitable growth matrix.

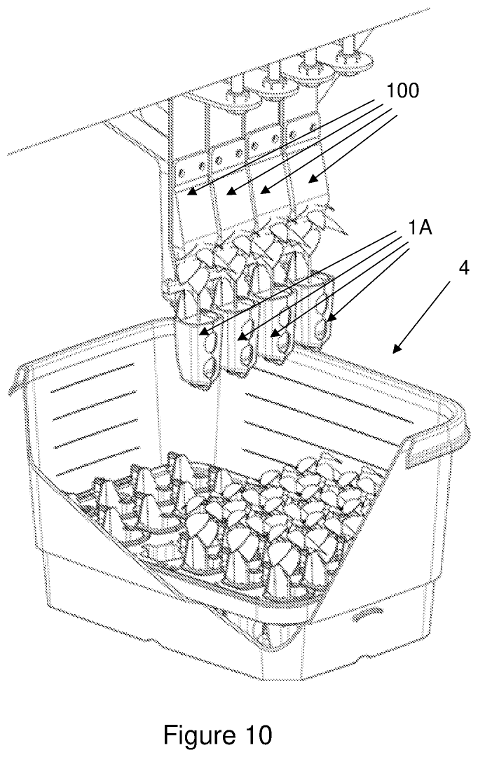

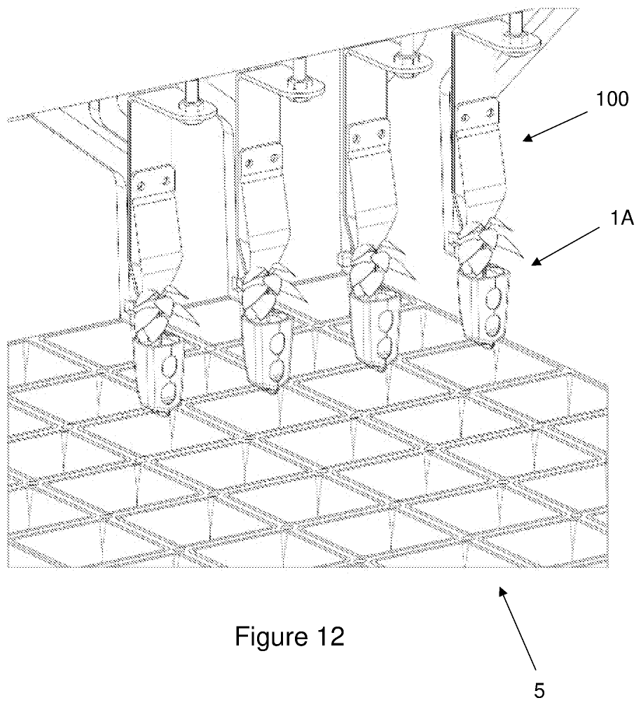

With reference to FIG. 10 and FIG. 12, it will be appreciated that the spacing of cells of tissue culture tray 4 differs from the spacing of cells of nursery tray 5. Actuation machinery of robot head 50, in conjunction with computing software and/or robotic sensors, can facilitate adjustment of the spacing of respective devices 100 and grippers 150 for alignment with cells of each of tray 4 and tray 5.

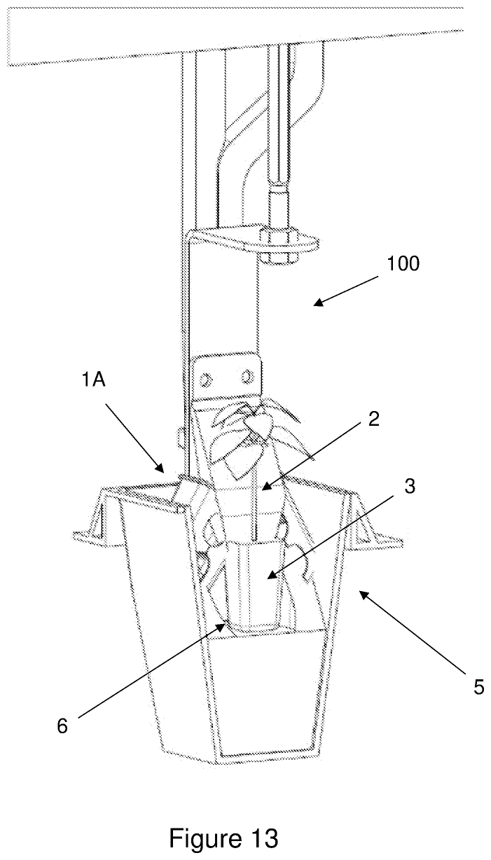

In use, with reference to FIG. 13, robotic arm 500 can move respective devices 100 into respective cells of nursery tray 5, and force respective plantlet holders 1 into the growth medium in the respective cells. Movement of plantlet holders 1 using robotic arm 500 and/or actuation machinery of robot head 50 can produce indentations or grooves or the like in the growth medium into which plantlets 2 can be ejected.

It will be appreciated that the production of indentations or grooves by forcing plantlet holders 1 into growth medium is optional. In some circumstances, it may be desirable to pre-dibble the growth medium using separate equipment. In some cases, it may be desirable to eject plantlets into the growth medium without dibbling or otherwise producing indentations or grooves.

In use, when plantlet holders 1 are to be opened and plantlets 2 ejected, each respective plantlet holder 1 is aligned with insertion body 140 of opener 110 of each respective device 100, by actuation of actuation machinery of robot head 50.

With respective plantlet holders 1 positioned in respective cells of nursery tray 5, the pneumatic actuators of robot head 50 are linearly actuated, resulting in linear actuation of actuator component 120 and blade component 130 and actuation of insertion body 140 connected thereto.

In use, linear actuation of actuator component 120 and blade component 130 moves insertion body 140 towards the respective plantlet holder 1. As insertion body 140 is moved towards plantlet holder 1, the insertion body guide in the form of fin 1405 of insertion body 140 is forced against a central end portion of gripper 150 formed by respective ends 1512 and 1522. Forcing of fin 1405 against the central end portion of gripper 150 guides insertion end 1401 forward somewhat as insertion body continues towards plantlet holder 1.

In use, continuing linear actuation of actuator component 120 and blade component 130 moves insertion end 1401 and insertion body 140 into open top 105 of body 10 of plantlet holder 1. As insertion end 1401 and insertion body 140 move into body 10 of plantlet holder 1, sides of insertion body 140 exert lateral force internally against left side 103 and right side 104 of body 10 of plantlet holder 1. Lateral force is initially exerted by insertion body 10 towards rear face 102 of plantlet holder 1. Continuing lateral force exerted by insertion body 140 within body 10 of plantlet holder 1 separates body 10 in a controlled fashion at the central seams and openings thereof.

In use, with body 10 fully separated, continuing linear actuation of actuator component 120 and blade component 130 moves insertion body 140 further forward under guide by fin 1405. Further forward movement of insertion body 140 pushes growth medium 3 and plantlet 2 away from device 100 and into growth medium within the cell of nursery tray 5, optionally into the groove or indentation in the growth medium where this has been produced e.g. using plantlet holder 1 under the control of system 1000.

In use, with plantlet 2 and growth medium 3 ejected into nursery tray 5, empty plantlet holder 1 can be moved away from nursery tray 5 and disposed using robotic arm 500 and robot head 50, such as by moving robot head to a plantlet holder disposal area and releasing separated plantlet holder 1 from gripper 150 (not shown). Alternatively, empty plantlet holder 1 can be returned to tissue culture tray 4, such as to its earlier position within tissue culture tray 4.

In use, robotic arm can then return to a position wherein respective devices 100 are over specific cells of specific tissue culture tray 4, wherein further plantlet holders 1 can be obtained for transfer of further plantlets 2 and growth medium 3 to further cells of nursery tray 5.

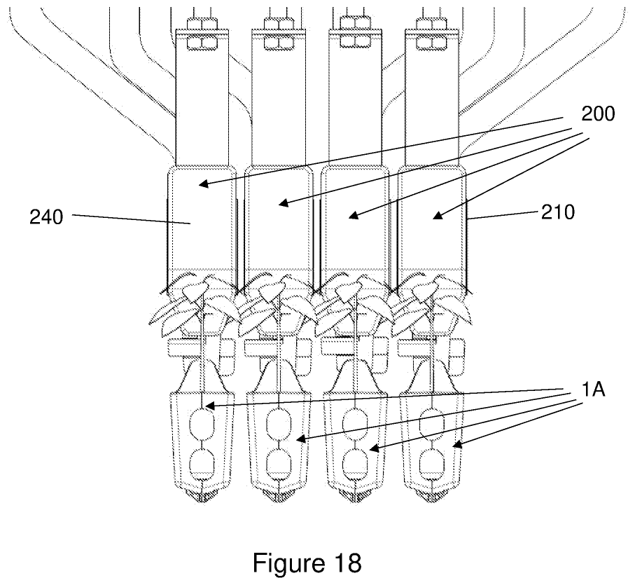

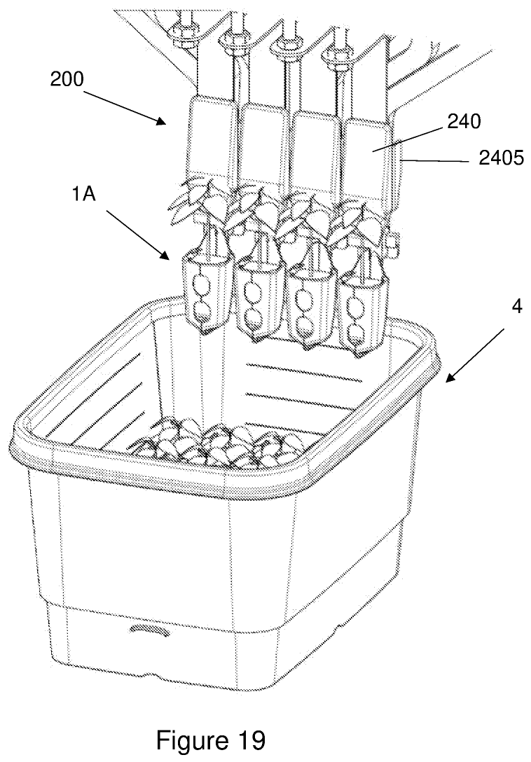

As hereinabove noted, description herein has primarily focused on one typical device embodiment, device 100. With reference to FIGS. 18 to 21, a further device embodiment, device 200 is shown.

It will be appreciated that insertion body 240 of device 200 is a somewhat different shape as compared to device 100. The most notable difference between device 200 and device 100 relates to the arrangement for guiding insertion body 240 relative to plantlet holder 1. In place of fin 1405 of device 100, device 200 comprise first and second lateral guide fins 2405.

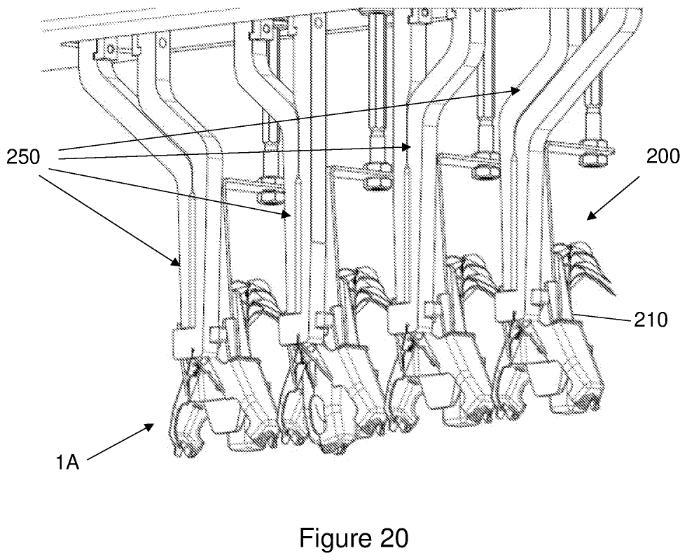

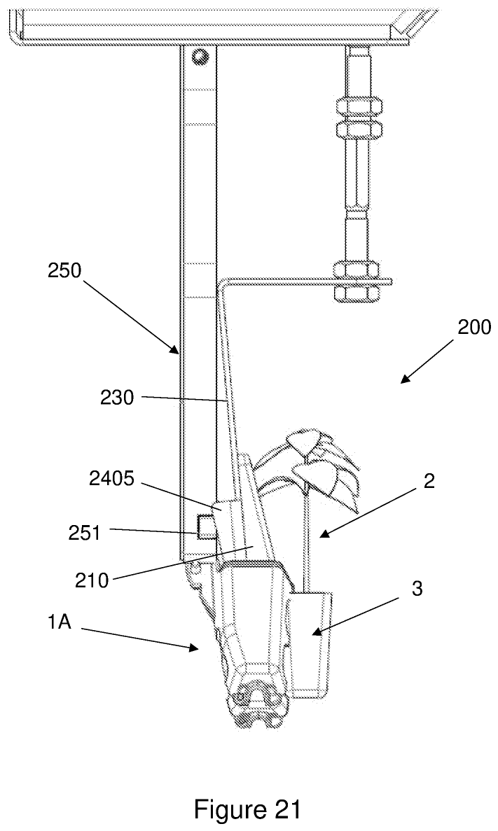

As best seen in FIGS. 20 and 21, device 200 is typically used in conjunction with gripper 250 comprising lateral projections 251, wherein forcing of lateral guide fins 2405 against lateral projections 251 guides insertion body 240 into body 10 of plantlet holder 1 in a similar manner as herein described in relation to device 100 and fin 1405 thereof.

Notably however, insertion body 240 is in fixed not hinged connection with blade component 230 thereof. Blade component 230 is a resiliently flexible blade component, such that forcing of lateral guide fins 2405 against lateral projections 251 of gripper 250 flexes blade component (and insertion body 240 fixed thereto) forward, rather than pivoting the insertion body away from the blade component as is the case for insertion body 140 of device 100.

With the above in mind, it will be appreciated that lateral guide fins 2405 may be of outer sides of insertion body 240 of device 200 or may alternatively extend from second or back face 2302 of blade component 230.

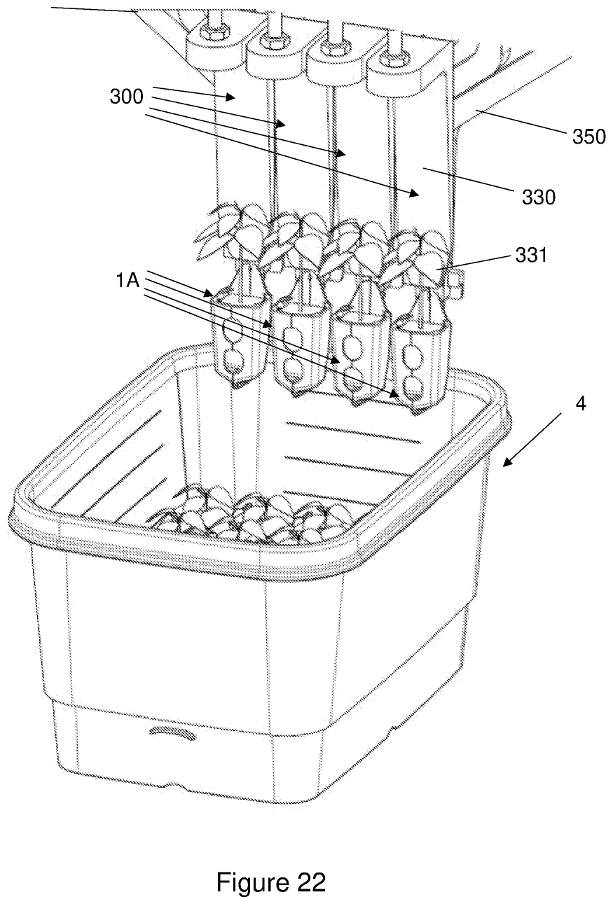

With reference to FIG. 22, a further device embodiment, device 300, is shown. Notably, device 300 does not include a separate insertion body such as insertion body 140 of device 100 and insertion body 240 of device 200. Rather, blade component 330 of device 300 is structured to function in a similar manner as the insertion bodies of the other device embodiments as described herein.

By way of elaboration, insertion end 331 is formed as part of blade component 330. Furthermore, and similar to blade component 230 of device 200, blade component 330 is a resiliently flexible blade component.

Another notable difference between device 300 and devices 100 and 200, is that device 300 does not include guides for insertion end 331. Rather, insertion end 331 is bent away from gripper 350 somewhat such that insertion end 331 is guided into body 10 of plantlet holder 1 secured by gripper 350 without the need for a guide component.

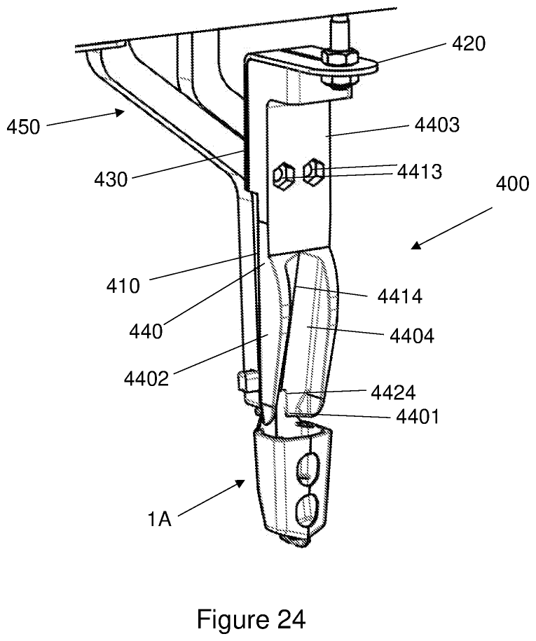

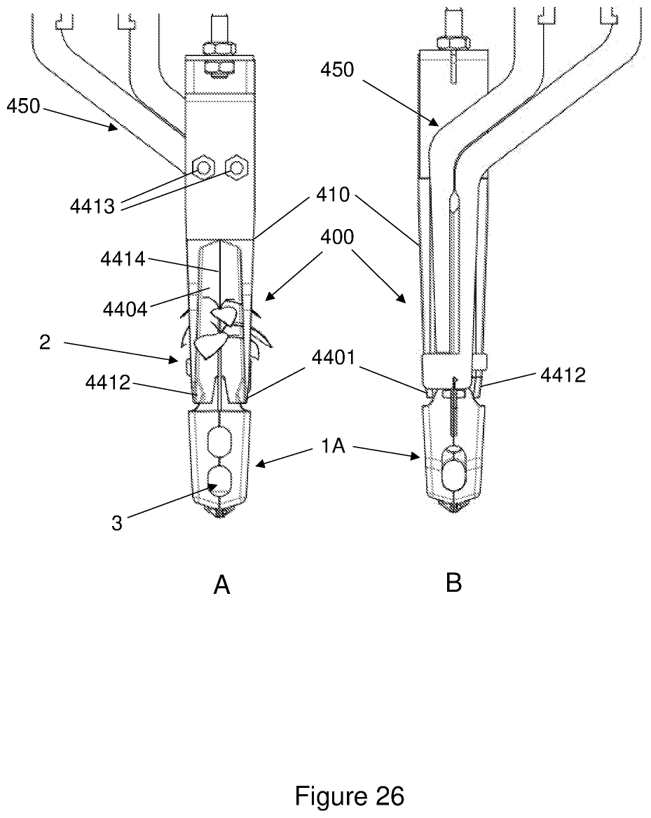

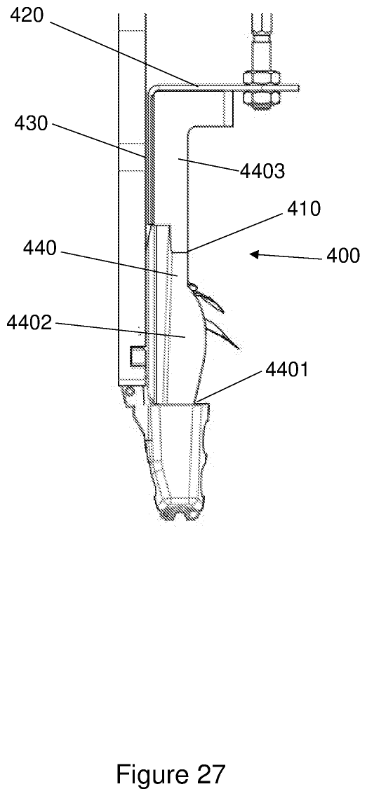

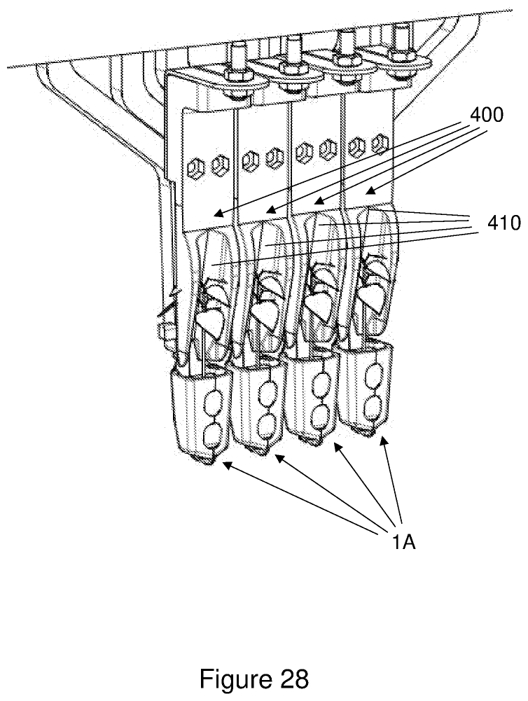

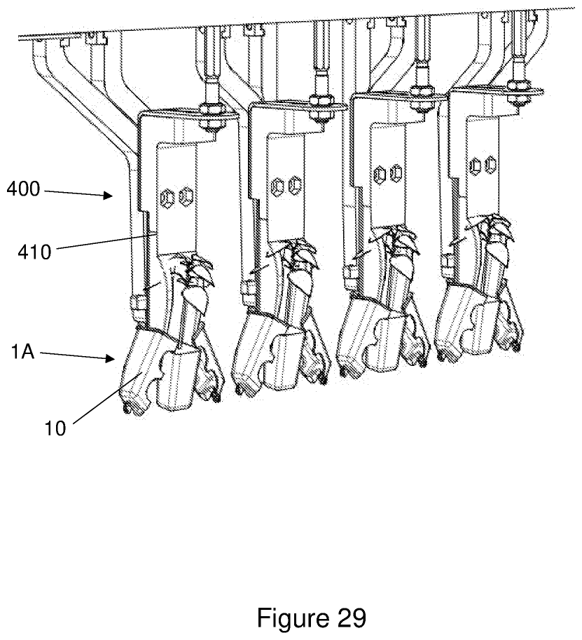

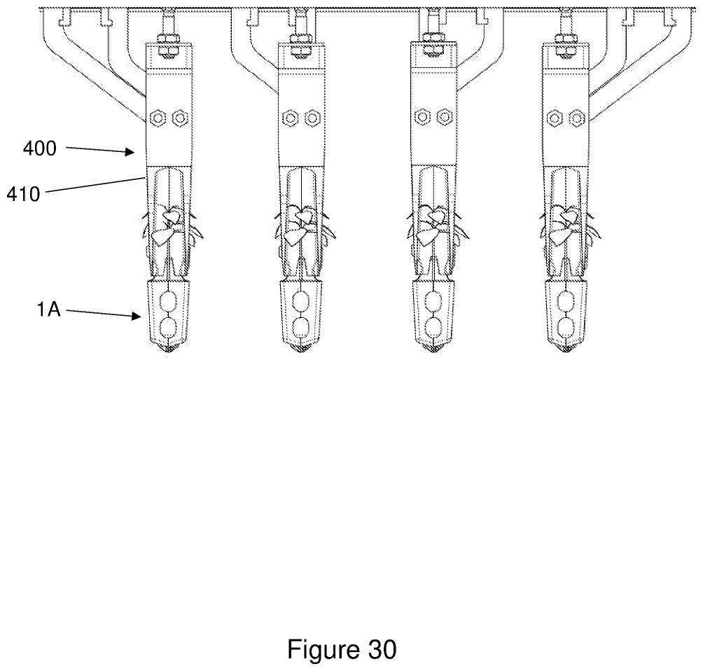

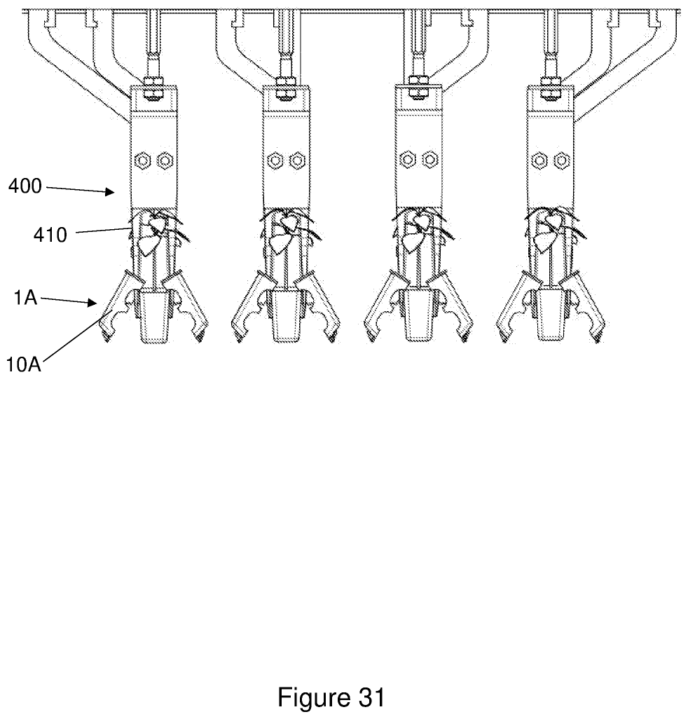

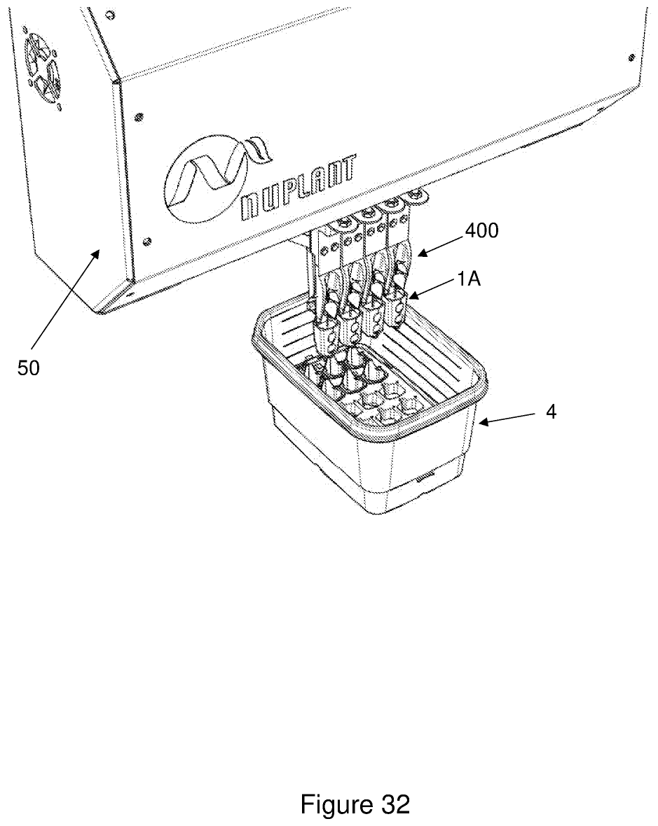

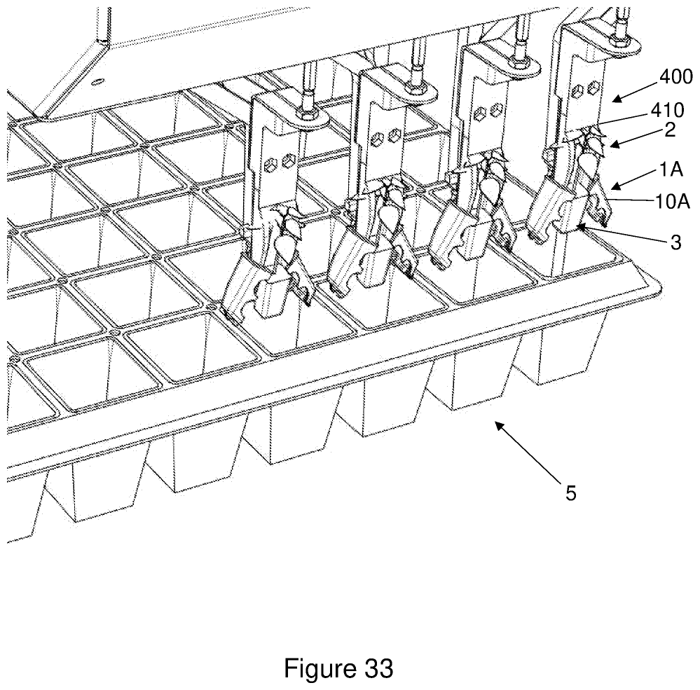

With reference to FIGS. 24 to 33, a further device embodiment, device 400, is shown. As discussed in further detail below, device 400 may be particularly advantageous or preferred in at least some instances.

Device 400 comprises opener 410. Opener 410 of device 400 is associated with gripper 450, in a substantially similar manner as hereinabove described in relation to opener 110 of device 100 and gripper 150. As for device 100, to avoid doubt, it will be appreciated that gripper 450 may, but need not, be considered part of device 400.

Opener 410 of device 400 comprises actuator component 420; blade component 430; and insertion body 440.

Similarly as for device 100 and device 200, actuator component 420 and blade component 430 of opener 410 are formed as an L-shaped bracket, wherein actuator component 420 comprises a horizontal portion of the bracket and blade component 430 comprises a vertical portion of the bracket. Notably, blade component 430 is approximately half the length of corresponding blade components of devices 100 and 200.

Insertion body 440 of opener 410 comprises insertion end 4401; first and second walls 4402; and support portion 4403. Insertion body 440 further comprises insertion face 4404 extending between first and second walls 4402.

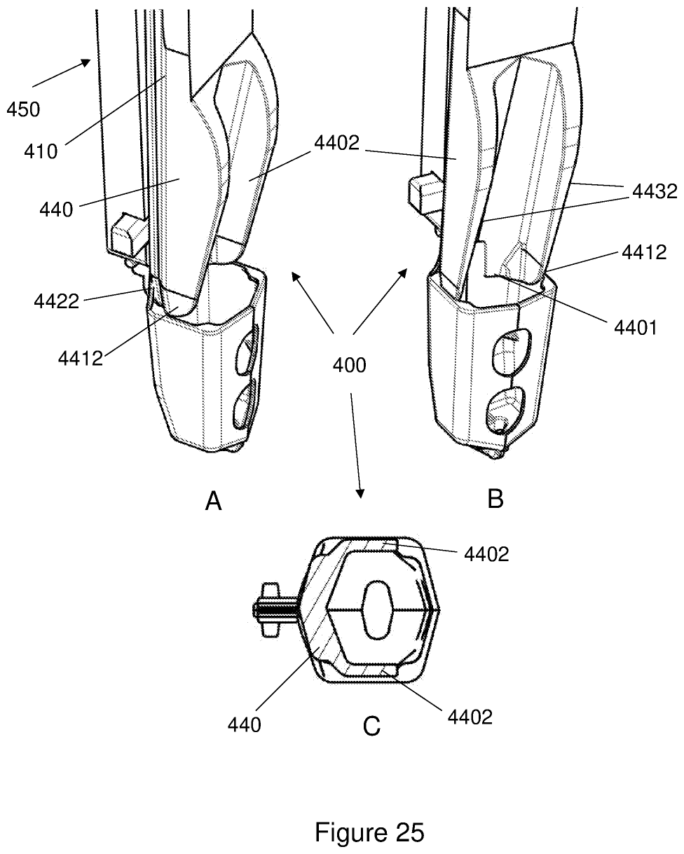

As best seen in FIG. 25, insertion end 4401 and insertion body walls 4402 of insertion body 440 are subtly tapered. Front corners 4412 and rear corners 4422 of insertion body walls 4402 taper inwardly towards insertion end 4401. To avoid doubt, tapered ends of insertion walls 4402 may be considered a part of insertion end 4401.

Also clearly seen in FIG. 25, insertion body walls 4402 of insertion body 440 comprise ramped edges 4432. Ramped edges 4432 ramp or angle down towards insertion end 4401.

Support portion 4403 of insertion body 440 is in the form of a substantially rigid L-shaped block fixed to the L-shaped bracket forming actuator component 420 and blade component 430. As shown in the figures, support portion 4403 is fixed using stud arrangement 4413, although any suitable fastening or fixing arrangement-including mechanical and/or chemical fasteners-may be used.

Insertion face 4404 of insertion body 440 is a slanted insertion face. More particularly, insertion face 4404 slants downwards from a position towards support portion 4403 to a position towards insertion end 4401. Additionally, insertion face 4404 slants inwardly from first and second insertion body walls 4402 towards central seam 4414. Insertion face 4404 is forked or pronged, comprising central substantially V-shaped gap or slot 4424 extending from insertion end 4401 to central seam 4414.

Similarly as hereinabove described in relation to device 100, in use, linear actuation of actuator component 420 of opener 410 moves insertion body 440 towards plantlet holder 1.

Notably, in contrast to devices 100 and 200, device 400 has no guide fins to be forced against gripper 450. Accordingly, in use, continuing linear actuation of actuator 420 forces insertion end 4401 of insertion body 440 directly into plantlet holder 1, without guidance against gripper 450.

As insertion end 4401 of insertion body 440 enters plantlet holder 1, initially, rear corners 4412 of insertion body walls 4402 engage with top 105 and rear face 102 of plantlet holder 1. Subsequently, as insertion body 440 is actuated further into plantlet holder 1, ramped edges 4432 of insertion body walls 4402 engage with top 105 of plantlet holder 1 at or towards front face 101. In the particular case of use with plantlet holder 1A as shown in FIGS. 24 to 33, rear corners 4412 of insertion body walls 4402 engage with corner bevels 1051A towards rear face 102A of plantlet holder body 10A, and ramped edges 4432 of insertion body walls 4402 engage with corner bevels 1051A towards front face 101A of plantlet holder body 10A.

As hereinabove described, blade component 430 is a truncated blade component, and support portion 4403, in the form of substantially rigid L-shaped block, is fixed to actuator component 420 and blade component 420. Accordingly, in use, as insertion body 440 is inserted into plantlet holder 1 opener 400 remains substantially rigid.

Similarly as hereinabove described in relation to device 100, in use, linear actuation of actuator component 420 of device 400 and engagement of insertion body 440 with plantlet holder 1 results in lateral force within body 10 of plantlet holder 1 against left side 103 and right side 104, wherein the lateral force within body 10 separates body 10 at the central seams thereof. Continuing linear actuation of actuator component 420 pushes growth medium 3 and plantlet 2 out from separated body 10 of plantlet holder 1.

In at least some circumstances, device 400 may be considered particularly preferred or advantageous. Notably, the substantially rigid arrangement of opener 410 may be particularly effective for consistent, controlled separation of plantlet holder 1 in use. Notably, the sloped arrangement of insertion face 4404; the pronged arrangement of insertion face 4404; and/or the presence of insertion body walls 4402, along or in combination, may advantageously assist with neat and controlled transfer of growth medium 3 and plantlet 2 from body 10.

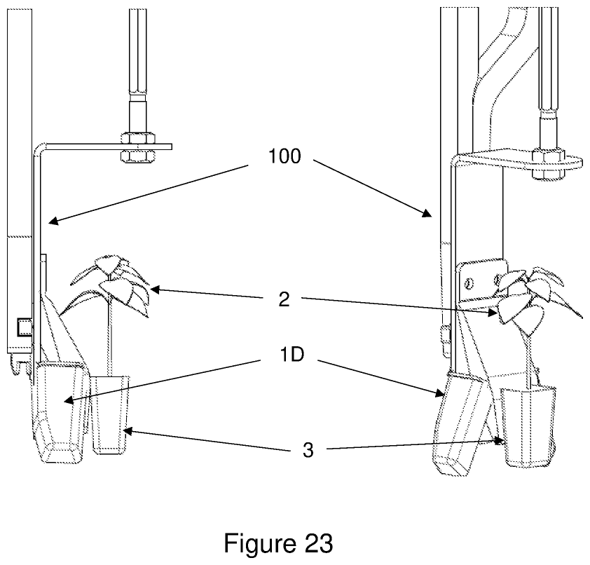

In the figures, operation of devices 100-400 has been illustrated primarily with reference to plantlet holder 1A. It will be appreciated, however, that similar operation occurs with plantlet holders 1B, 1C, and 1D as hereinabove described. In the particular case of plantlet holder 1D, opening of this plantlet holder and ejection of plantlet 2 and agar plug 3 is shown in FIG. 23. It will further be appreciated that plantlet holders, such as plantlet holder 1, can be modified or adjusted to be specifically compatible with one or more of devices 100-400 as described herein. By way of non-limiting example, with reference to FIG. 25, it will be appreciated that insertion end 4401, and walls 4402 more generally, of insertion body 440 are shaped to neatly engage with top 105A of plantlet holder 1A, including corner bevels 1051A.

Exemplary use of devices with robotic systems for transfer of tissue cultured plantlets to nursery-suitable material has been described with primary reference to device 100. It will be understood that devices 200, 300, and 400 may be used with robotic systems in the same or a similar manner. In some circumstances, it may be particularly preferred or advantageous to use device 400 with robotic systems for transfer of tissue cultured plantlets to nursery-suitable material.

Devices, systems, and methods herein have been described with primary reference to robotic arrangements included a pneumatic robot tool head attached to a robotic arm. To avoid doubt, various other robotic arrangements may be suitable in the context of at least certain aspects and embodiments of the invention. By way of non-limiting example, in some cases, electronic or hydraulic, including microhydraulic, actuators may be suitable or desirable for operating openers and/or grippers as described herein. Furthermore, robot tool heads and the like may be used in conjunction with e.g. pulley and motor mechanisms in place of a robotic arms, such pulley and motor mechanisms in some cases controlled by Programmable Logic Controller (PLC) arrangements.

Devices, systems, and methods herein have been described with primary reference to tissue culture plantlets, tissue culture plantlet holders, and tissue culture trays, used in conjunction with nursery trays. To avoid doubt, subject matter encompassed by the invention is not limited to tissue culture plantlets, nursery regeneration, or transfer from tissue culture to nursery trays, although aspects and embodiments of the invention may have particular benefits in these contexts.

It will also be appreciated that, unless the context requires otherwise, “tissue culture” is to be broadly interpreted herein to encompass not only traditional tissue culture techniques, but also emerging techniques (inclusive of modern in vitro micropropagation techniques) that may be considered modern variations of tissue culture or ‘tissue-culture like’. For a recent overview of plant tissue culture, the skilled person is directed to Smith, Roberta H. Plant tissue culture: techniques and experiments. Academic Press, 2012, incorporated herein in full by reference. For a recent overview of current and emerging techniques for plant micropropagation, the skilled person is directed to Cardoso, Jean Carlos, Lee Tseng Sheng Gerald, and Jaime A. Teixeira da Silva. “Micropropagation in the twenty-first century.” Plant cell culture protocols (2018): 17-46, incorporated herein in full by reference.

Furthermore, aspects and embodiments of the invention may have applications for, without limitation, greenhouse cultivation, indoor gardening, or hydroponic cultivation. That is, it is to be understood that the subject matter of the invention is not necessarily limited to applications involving sterile and/or clonal plant culture.

The skilled person will appreciate that a range of materials can be suitable for construction of devices and systems, and components thereof, as described herein. Typically, suitable plastic and/or metallic materials are used, as are well-known in the field of robotics. In typical embodiments, blade and actuator components of devices 100-400 as described herein are formed from or comprise a suitable metal, such as aluminium or stainless steel. In typical embodiments, insertion body components of devices 100-400 as described herein are formed from or comprise a suitable plastic, e.g. thermoplastics such as acrylics, nylons, or polycarbonates although without limitation thereto. In relation to plantlet holders such as plantlet holder 1, typical materials are discussed in WO2011/014933 and WO2012/167332 incorporated herein.

It will be understood generally that the above description of embodiments of the invention is provided for purposes of description to one of ordinary skill in the related art. It is not intended to be exhaustive or to limit the invention to a single disclosed embodiment. In some instances, well-known components and/or processes have not been described in detail, so as not to obscure the embodiments described herein.

As described, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. The invention is intended to embrace all alternatives, modifications, and variations that have been discussed herein, and other embodiments that fall within the spirit and scope of the invention.

In this specification, the use of the terms “suitable” and “suitably”, and similar terms, is not to be read as implying that a feature or step is essential, although such features or steps may well be preferred.

In this specification, the indefinite articles “a” and “an” are not to be read as singular indefinite articles or as otherwise excluding more than one or more than a single subject to which the indefinite article refers. For example, “an” opener includes one opener, one or more openers, and a plurality of openers.

In this specification, the terms “comprises”, “comprising”, “includes”, “including”, and similar terms, are intended to denote the inclusion of a stated integer or integers, but not necessarily the exclusion of another integer or other integers, depending on context. That is, a product, composition, or method, etc., that comprises or includes stated integer(s) need not have those integer(s) solely, and may well have at least some other integers not stated, depending on context.

In this specification, the terms “consisting essentially of” and “consists essentially of” are intended to mean a non-exclusive inclusion only to the extent that, if additional elements are included beyond those elements recited, the additional elements do not materially alter basic and novel characteristics. That is, an apparatus, system, or method that “consists essentially of” one or more recited elements includes those elements only, or those elements and any additional elements that do not materially alter the basic and novel characteristics of the apparatus, system, or method.

In this specification, terms such as “above” and “below”; “front” and “back”; “top” and “bottom”; “left” and “right”; “horizontal” and “vertical”, and the like, may be used for descriptive purposes. However, it will be understood that embodiments can potentially be arranged in various orientations, and that such relative terms are not limiting and may be interchangeable in appropriate circumstances.

In this specification, unless the context requires otherwise, the terms “connection”, “connected”, “connecting”, and the like, are not to be read as limited to direct connections and may also include indirect connections. For example, unless the context requires otherwise, a stated first component “connected” to a stated second component may be connected via, through, or by, one or more unstated components.

1. A device for robotic transfer of a plantlet from a plantlet holder, the device comprising an opener for opening of the plantlet holder wherein the opener comprises: an actuator component for connection to a robotic tool head; and a manipulation component comprising an insertion end for manipulating the plantlet holder.

2. (canceled)

3. The device of claim 1, wherein the insertion end is a thinned and/or tapered insertion end.

4. The device of claim 1, wherein the opener comprises a blade component.

5. The device of claim 4, wherein the blade component is of or connected to the actuator component.

6. The device of claim 1, wherein the insertion end is of an insertion body.

7. The device of claim 6, wherein the insertion body is connected to the blade component.

8. The device of claim 7, wherein the insertion body is in fixed connection with the blade component.

9. The device of claim 1, wherein the opener is substantially rigid.

10. The device of claim 7, wherein the insertion body is in pivotal connection with the blade component.

11. The device of claim 1, wherein the insertion body comprises one or more insertion body walls.

12. The device of claim 1, comprising a plantlet holder securing component, wherein the securing component comprises a gripper for gripping the plantlet holder, the gripper connectable to the robotic tool head, wherein the gripper is actuatable between a gripping configuration and a releasing configuration.

13-14. (canceled)

15. The device of claim 12, wherein a central or longitudinal axis of the gripper is substantially alignable to a central or longitudinal axis of the opener.

16. The device of claim 12, wherein linear actuation of the actuator component of the opener of the device is adapted to apply force from the manipulation or insertion portion of the opener to the plantlet holder gripped by the gripper of the device, to open the plantlet holder.

17. The device of claim 12, comprising one or more guides for guiding the opener, or a portion thereof, relative to the plantlet holder when held by the gripper.

18. (canceled)

19. The device of claim 16, wherein the one or more guides comprise one or more protrusions for contacting the gripper of the device to guide the insertion portion of the opener relative to the plantlet holder, wherein the one or more protrusions are ramped or angled protrusions wherein the actuator portion of the opener forces the one or more ramped protrusions against the gripper such that the insertion portion of the opener is forced away from the gripper and guided relative to the plantlet holder held by the gripper.

20-22. (canceled)

23. The device of claim 12, wherein:
the opener comprises an actuator component for connection to the robot tool head and a tapered insertion portion connected to the actuator, wherein the tapered insertion portion is for insertion into the body of the plantlet holder by actuation of the actuator portion, to separate the plantlet holder body along the one or more central seams and/or central openings thereof, and
the gripper comprises first and second fingers for connection to the robot tool head, wherein the gripper is actuatable between a gripping configuration wherein gripping portions of the first and second fingers are together for securing the handling portion of the plantlet holder, and a releasing configuration wherein gripping portions of the first and second fingers are apart for releasing the handling portion of the plantlet holder.

24. (canceled)

25. The device of claim 23, comprising a guide for guiding the opener against the gripper for opening of the plantlet holder, wherein the guide comprises at least one protrusion for contacting the gripping portion of the first and/or second fingers of the gripper, wherein the actuation of the actuator portion of the opener forces the protrusion against the gripping portion to guide the insertion portion of the opener away from the gripper for insertion into the body of the plantlet holder.

26. (canceled)

27. A robotic system comprising a robot tool head comprising the device of claim 1 connected to a robot arm.

28-29. (canceled)

30. A method of robotically transferring a plantlet from a plantlet holder, including a step of opening the plantlet holder with the device of claim 1.

31. (canceled)
